(19) United States
(12) Patent Application Publication
(10) Pub. No.: US 2023/0200704 A1
(43) Pub. Date: Jun. 29, 2023

(54) ELECTROCARDIOGRAM ELECTRODES PACKAGING

(71) Applicant: LDC BUSINESS CONSULTING LLC, Temple, TX (US)

(72) Inventor: Lance Cromwell, Temple, TX (US)

(21) Appl. No.: 18/147,597

(22) Filed: Dec. 28, 2022

Related U.S. Application Data
(60) Provisional application No. 63/294,739, filed on Dec. 29, 2021.

(51) Int. Cl. A61B 5/263; A61B 5/28

(52) U.S. Cl. CPC A61B 5/263; A61B 5/28

(57) ABSTRACT

An electrocardiogram (ECG) electrode warming system with a warmer and at least one ECG electrode. The warmer has an exothermic warming composition. The at least one ECG electrode is configured to attach to a patient's body to detect electrical signals and is thermally connected to the warmer. An insert may be positioned between the at least one ECG electrode and the warmer. The at least one ECG electrode may be removably connected to the insert and the warmer may be bonded to the insert with an adhesive. A barrier may be configured to separate the exothermic warming composition from a reactant composition. The barrier is breakable by a user to allow the reactant composition to contact the exothermic warming composition. Contact between the reactant composition and the exothermic warming composition causes the exothermic warming composition to release heat.

### CROSS REFERENCE TO RELATED APPLICATIONS

This application claims the benefit of the filing date of U.S. Provisional Patent Application 63/294,739 entitled “Electrocardiogram Electrodes Packaging” to Lance Cromwell that was filed on Dec. 29, 2021, the disclosure of which is hereby incorporated herein by this reference.

### TECHNICAL FIELD

Aspects of this document relate generally to electrocardiogram (ECG) electrodes, and more specifically to packaging for ECG electrodes.

### BACKGROUND

Undergoing any medical procedure often causes the patient to experience anxiety and discomfort prior to or during the procedure. This anxiety and discomfort are exacerbated by any conditions which negatively affect the patient's body. For example, if the patient is uncomfortably cold, this can affect how the patient is feeling about the procedure.

One common source of discomfort and shock is when a medical professional places electrocardiogram (ECG) electrodes on the patient. Because operating rooms and other locations within a hospital are kept at around 68 degrees, ECG electrodes tend to be very cold and cause the patient to tense up on contact due to the unexpected temperature change. This may increase the anxiety, discomfort, and apprehension of the patient.

### SUMMARY

Aspects of this document relate to an electrocardiogram (ECG) electrode warming system comprising a warmer having an exothermic warming composition, at least one ECG electrode configured to attach to a patient's body to detect electric signals, wherein the at least one ECG electrode is thermally connected to the warmer, an insert positioned between the at least one ECG electrode and the warmer, wherein the at least one ECG electrode is removably connected to the insert and the warmer is bonded to the insert with an adhesive, and a barrier configured to separate the exothermic warming composition from a reactant composition, wherein the barrier is breakable by a user to allow the reactant composition to contact the exothermic warming composition and wherein contact between the reactant composition and the exothermic warming composition causes the exothermic warming composition to release heat.

Particular embodiments may comprise one or more of the following features. The warmer may be configured to warm the at least one ECG electrode to at least a selected temperature within five minutes and maintain the at least one ECG electrode at or above the selected temperature for at least 45 minutes. The selected temperature may be 120 degrees Fahrenheit. The exothermic warming composition may be packaged within a fabric with a weight of at least 50 grams per square meter.

Aspects of this document relate to an electrocardiogram (ECG) electrode warming system comprising a warmer having an exothermic warming composition, at least one ECG electrode configured to attach to a patient's body to detect electric signals, wherein the at least one ECG electrode is thermally connected to the warmer, and a barrier configured to separate the exothermic warming composition from a reactant composition, wherein the barrier is breakable by a user to allow the reactant composition to contact the exothermic warming composition and wherein contact between the reactant composition and the exothermic warming composition causes the exothermic warming composition to release heat.

Particular embodiments may comprise one or more of the following features. The warming system may further comprise an insert positioned between the at least one ECG electrode and the warmer, wherein the at least one ECG electrode is removably connected to the insert. The warmer may be bonded to the insert. The warmer may be bonded to the insert with an adhesive. The barrier may be an envelope configured to encase the warmer and the reactant composition is ambient air. The warmer may be configured to warm the at least one ECG electrode to at least a selected temperature within five minutes and maintain the at least one ECG electrode at or above the selected temperature for at least 45 minutes. The selected temperature may be 120 degrees Fahrenheit. The exothermic warming composition may be packaged within a fabric with a weight of at least 50 grams per square meter.

Aspects of this document relate to an electrocardiogram (ECG) electrode warming system comprising a warmer having an exothermic warming composition and at least one ECG electrode configured to attach to a patient's body to detect electric signals, wherein the at least one ECG electrode is removably and thermally connected to the warmer.

Particular embodiments may comprise one or more of the following features. The exothermic warming composition may be air-activated. The ECG electrode warming system may further comprise a barrier configured to separate the exothermic warming composition from a reactant composition, wherein the barrier is breakable by a user to allow the reactant composition to contact the exothermic warming composition. The barrier may be an envelope configured to encase the warmer and the reactant composition may be ambient air. Contact between a reactant composition and the exothermic warming composition may cause the exothermic warming composition to release heat. The ECG electrode warming system may further comprise an insert positioned between the at least one ECG electrode and the warmer, wherein the at least one ECG electrode is removably connected to the insert and the warmer is bonded to the insert. The warmer may be configured to warm the at least one ECG electrode to at least a selected temperature within five minutes. The warmer may be configured to maintain the at least one ECG electrode at or above a selected temperature for at least 45 minutes.

The foregoing and other aspects, features, applications, and advantages will be apparent to those of ordinary skill in the art from the specification, drawings, and the claims. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U. S.C. § 112(f) are sought to be invoked to define the inventions, the claims will specifically and expressly state the exact phrases “means for” or “step for”, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U. S.C. § 112(f). Moreover, even if the provisions of 35 U.S.C. § 112(f) are invoked to define the claimed aspects, it is intended that these aspects not be limited only to the specific structure, material or acts that are described in the preferred embodiments, but in addition, include any and all structures, materials or acts that perform the claimed function as described in alternative embodiments or forms of the disclosure, or that are well known present or later-developed, equivalent structures, material or acts for performing the claimed function.

The foregoing and other aspects, features, and advantages will be apparent to those of ordinary skill in the art from the specification, drawings, and the claims.

### BRIEF DESCRIPTION OF THE DRAWINGS

Implementations will hereinafter be described in conjunction with the appended drawings, where like designations denote like elements, and:

FIG. 1 is a perspective view of an electrocardiogram (ECG) electrode warming system;

FIG. 2 is a perspective view of the warmer, insert, and electrodes of the ECG electrode warming system shown in FIG. 1;

FIG. 3 is an exploded view of the ECG electrode warming system shown in FIG. 1;

FIG. 4 is a top view of an electrode of the ECG electrode warming system shown in FIG. 1;

FIG. 5 is a cross section view of the electrode shown in FIG. 4 taken along line 5-5;

FIG. 6 is process diagram illustrating a method of warming an ECG electrode using the system shown in FIGS. 1-5; and

FIG. 7 is a process diagram illustrating a method of assembling the ECG electrode warming system shown in FIGS. 1-5.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of implementations.

### DETAILED DESCRIPTION

This disclosure, its aspects and implementations, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

While this disclosure includes a number of implementations that are described in many different forms, there is shown in the drawings and will herein be described in detail particular implementations with the understanding that the present disclosure is to be considered as an exemplification of the principles of the disclosed methods and systems, and is not intended to limit the broad aspect of the disclosed concepts to the implementations illustrated.

In the following description, reference is made to the accompanying drawings which form a part hereof, and which show by way of illustration possible implementations. It is to be understood that other implementations may be utilized, and structural, as well as procedural, changes may be made without departing from the scope of this document. As a matter of convenience, various components will be described using exemplary materials, sizes, shapes, dimensions, and the like. However, this document is not limited to the stated examples and other configurations are possible and within the teachings of the present disclosure. As will become apparent, changes may be made in the function and/or arrangement of any of the elements described in the disclosed exemplary implementations without departing from the spirit and scope of this disclosure.

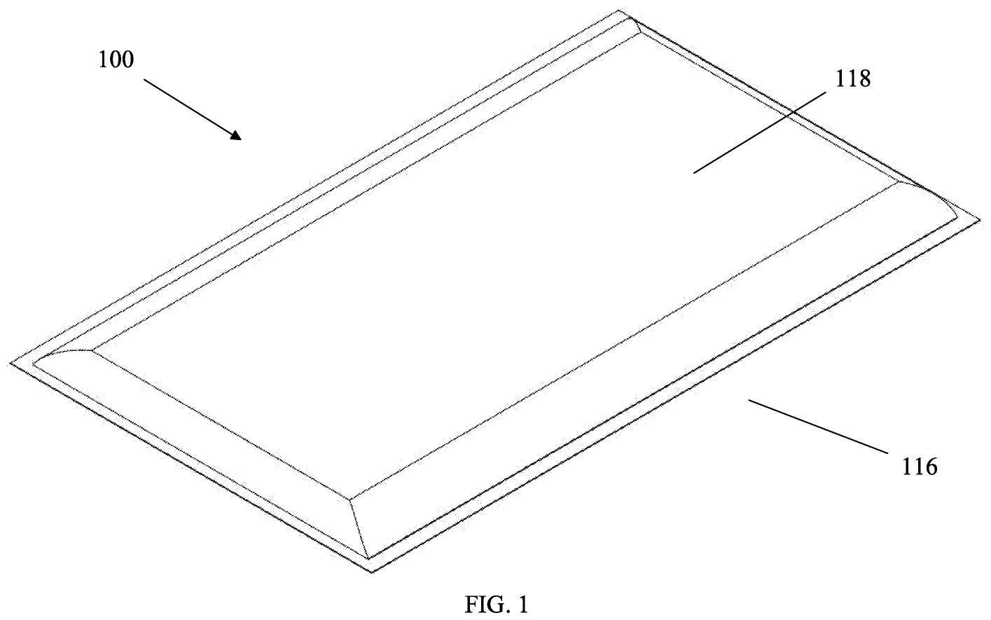

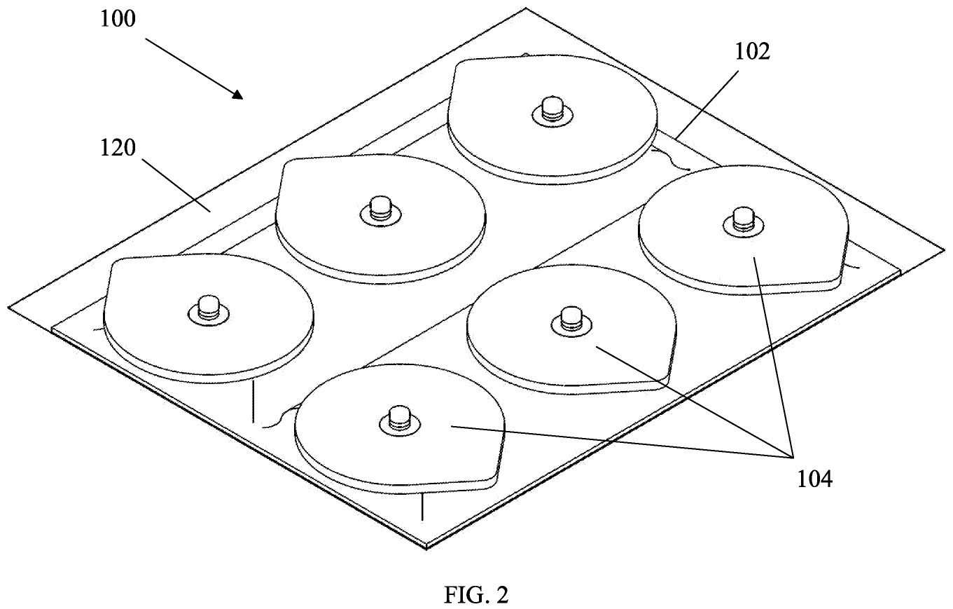

The present disclosure is related to an electrocardiogram (ECG) electrode warming system 100. The warming system 100 comprises a warmer 102 and ECG electrodes 104 package together, as shown in FIGS. 1-3. Any number of ECG electrodes 104 may be included in the system 100. For example, the warming system 100 may have at least one ECG electrode 104, at least two ECG electrodes 104, or a plurality of ECG electrodes 104. In a particular embodiment, the warming system 100 has six ECG electrodes 104. The ECG electrodes 104 are configured to attach to a patient's body to detect electrical signals. For example, the ECG electrodes 104 may be used to detect electrical signals generated by the patient's heart or brain.

The warming system 100 is designed and configured to warm up ECG electrodes 104 prior to placing them on the patient's body. This promotes patient health by keeping the patient comfortable, rather than shocking the patient's body with cold electrodes 104. This in turn improves the outcome of the medical procedure. For example, an ECG is affected by movement of the patient, even slight movement such as shivering. The more still the patient is, the more accurate the ECG results are. By helping the patient to be more relaxed and warmer, the warmed ECG electrodes 104 increase the accuracy of the ECG results.

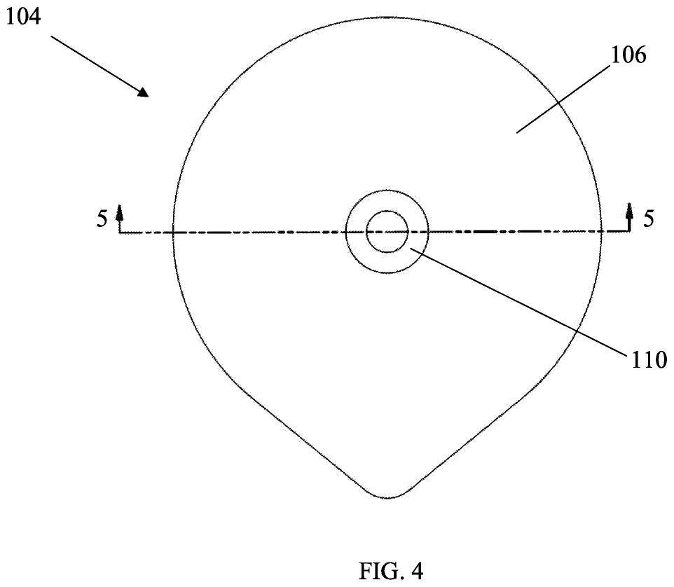

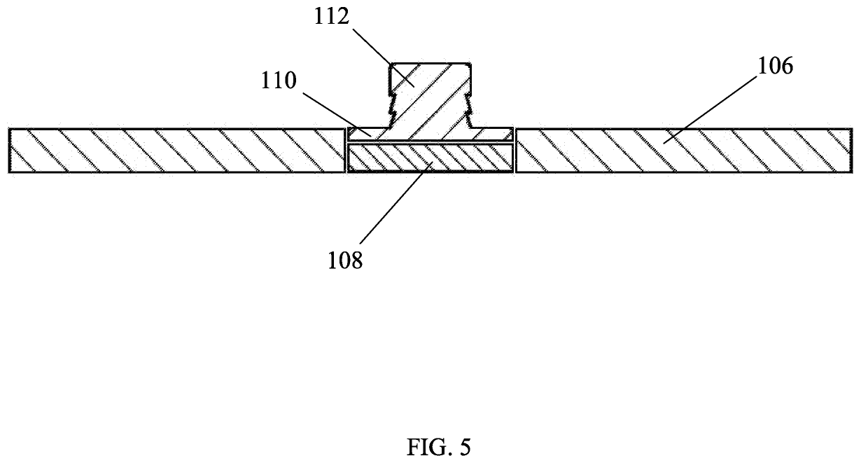

The ECG electrodes 104 may be removably and/or thermally connected to the warmer 102. This allows the ECG electrodes 104 to be easily detached from the warmer 102 to be applied to a patient's body while keeping the ECG electrodes 104 close to the warmer 102 so that the warmer 102 can effectively warm the ECG electrodes 104. Each ECG electrode 104 may have an adhesive pad 106, a sponge 108, and a snap 110, as shown in FIGS. 4-5. The adhesive pad 106 may have a cavity in its center and the sponge 108 may be positioned within the cavity of the adhesive pad 106 and may be soaked in a conductive gel such as a silver chloride gel. In some embodiments, the ECG electrode 104 may not have a sponge 108, and instead, the cavity may be filled with gel. The snap 110 is conductive and is in contact with the sponge 108 or the gel. The snap 110 also has a protrusion 112 extending away from the cavity. An external snap, such as on the end of a wire (not shown), may couple with the protrusion 112 and carry a signal entering the electrode 102 through the sponge 108 or the gel away from the electrode 104 to be measured.

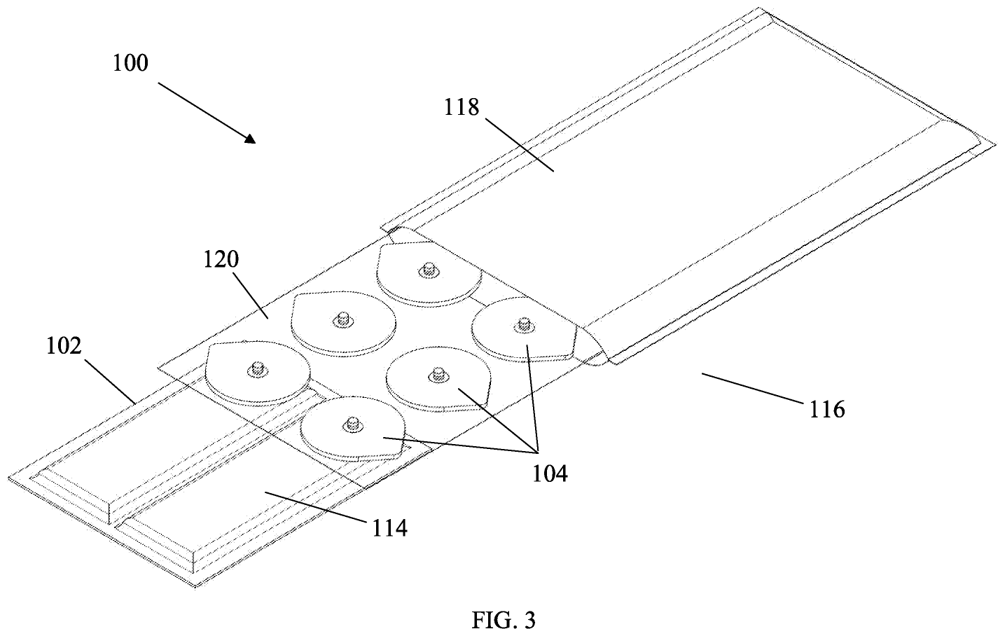

Returning to FIG. 3, the warmer 102 comprises an exothermic warming composition 114. The exothermic warming composition 114 may be any composition that undergoes an exothermic process to release heat and warm its surroundings. To control the timing of when the heat is released, the warming composition 114 may be separated or isolated from a reactant composition 116 that is configured to initiate and perpetuate the exothermic process. For example, the warming composition 114 may be air-activated and the reactant composition 116 may be ambient air. By separating the air-activated warming composition 114 from ambient air, the exothermic process experienced by the warming composition 114 may be paused until desired.

The warming system 100 may comprise a barrier 118 to separate the warming composition 114 from the reactant composition 116. In some embodiments, the barrier 118 is breakable by a user. Once broken, the barrier 118 no longer separates the warming composition 114 from the reactant composition 116 and contact between the reactant composition 116 and the warming composition 114 is allowed. As explained above, this causes the exothermic process to take place, releasing heat from the warming composition 114.

The barrier 118 may have any shape. For example, the warming composition 114 and the reactant composition 116 may be packaged together, with the barrier 118 extending between them as a sheet. Once this sheet is broken, the warming composition 114 and the reactant composition 116 are able to mix within the packaging, initiating the exothermic process. As another example, the barrier 118 may be the packaging itself, such as an envelope that encases the warmer 102 and the warming composition 114, as shown in FIGS. 1 and 3. The reactant composition 116 may be positioned outside of the barrier 118 so that, when the envelope is opened, the warming composition 114 and the reactant composition 116 can mix. In embodiments where the warming composition 114 is air-activated, ambient air contacts the warming composition 114 when the envelope is opened, initiating the exothermic process.

The exothermic process may be an oxidation process. Thus, the warming composition 114 may oxidize to release heat and warm the ECG electrode 104. The warming composition 114 may include ingredients such as iron, salt, water, activated charcoal, vermiculite, and/or saw dust. The warming composition 114 may be enclosed in an outer layer formed of a material that is permeable to the reactant composition 116. Additionally, the warming composition 114 may be enclosed in an outer layer formed of a material that helps prevent the warming composition 114 from passing through the outer layer. Heavier fabrics may help to prevent the warming composition 114 from passing through the outer layer, while remaining permeable to the reactant composition 116. Thus, the warming composition 114 may be packaged within an outer layer formed of a fabric with a weight of at least 50 grams per square meter (GSM). In some embodiments, the fabric may have a weight of at least 60 GSM, 70 GSM, 75 GSM or 80 GSM. In particular embodiments, the warming composition 114 is packaged within a polypropylene fabric. Other materials may also be implemented.

Some embodiments of the warming system 100 comprise an insert 120. The insert 120 provides a surface to which the ECG electrodes 104 can be removably attached. The insert 120 may be formed of a clear plastic material and may be a thin sheet of material. The insert 120 has a first side and a second side opposite the first side. The ECG electrodes 104 are attached to the first side. In some embodiments, the warmer 102 may be bonded to the second side of the insert 120. This may help to keep the warmer 102 in thermal contact with the ECG electrodes 104. In some embodiments, an adhesive is used to bond the warmer 102 to the insert 120. The adhesive may be any adhesive known in the art, and may be rolled onto the insert 120 or sprayed onto the insert 120. Other methods of applying the adhesive may also be implemented. In some embodiments, the adhesive is a polymer adhesive, an acrylic adhesive, a hot melt adhesive, or a resin adhesive. In a particular embodiment, the adhesive is a polyurethane glue. Other methods of bonding the warmer 102 to the insert 120 may also be implemented, such as using tape, double-sided tape, clips, rivets, staples, and the like.

The warming system 100 is configured to warm the ECG electrodes 104 to a comfortable temperature so that, when applied to the patient's body, the ECG electrodes 104 are not cold or uncomfortable. The warmer 102 may be configured to warm the ECG electrodes 104 to a selected temperature within a first time period. Additionally, the warmer 102 may be configured to maintain the ECG electrodes 104 at or above the selected temperature for a second time period. These two time periods help the user to know that the ECG electrodes 104 will be sufficiently warm during a specific time period, providing flexibility to the user if the exact moment when the electrodes 104 will be applied is unknown.

For some embodiments, the first time period is five minutes, and the warmer 102 is configured to warm the ECG electrodes 104 to the selected temperature within five minutes. For some embodiments, the first time period is ten minutes, and the warmer 102 is configured to warm the ECG electrodes 104 to the selected temperature within ten minutes. Other time periods may also be selected for the first time period. For some embodiments, the second time period is at least 45 minutes, and the warmer 102 is configured to maintain the ECG electrodes 104 at or above the selected temperature for at least 45 minutes. For some embodiments, the second time period is at least 90 minutes, and the warmer 102 is configured to maintain the ECG electrodes 104 at or above the selected temperature for at least 90 minutes. For some embodiments, the second time period is at least 120 minutes, and the warmer 102 is configured to maintain the ECG electrodes 104 at or above the selected temperature for at least 120 minutes. Other time periods may also be selected for the second time period. The selected temperature may be 110 degrees Fahrenheit, 120 degrees Fahrenheit, 130 degrees Fahrenheit, 140 degrees Fahrenheit, or any other temperature. It is desirable for the selected temperature to be high enough that the ECG electrodes 104 are warm to the touch for the patient.

In a particular embodiment of the warming system 100, if the ECG electrodes 104 begin at 70 degrees Fahrenheit, the warmer 102 is configured to warm the ECG electrodes 104 to 85 degrees Fahrenheit within one minute, 100 degrees Fahrenheit within two minutes, 110 degrees Fahrenheit within three minutes, 120 degrees Fahrenheit within four minutes, 130 degrees Fahrenheit within five minutes, 135 degrees Fahrenheit within six minutes, 140 degrees Fahrenheit within seven minutes, 145 degrees Fahrenheit within eight minutes, and/or 150 degrees Fahrenheit within nine minutes. The warmer 102 may be configured to warm the ECG electrodes 104 to 145 degrees Fahrenheit within 10 minutes, 150 degrees Fahrenheit within 11 minutes, and/or 155 degrees Fahrenheit within 12 minutes.

The warming system 100 may also be assembled as a kit. In such an embodiment, the separate components of the warming system 100 may be packaged together, but not assembled to the same degree as described above. For example, the kit may comprise at least one ECG electrode 104 and a warmer 102 as described above, the kit may comprise at least one ECG electrode 104, a warmer 102, and an insert 120, or the kit may comprise at least one ECG electrode 104, a warmer 102, an insert 120, and an adhesive, but these components may not be coupled together in the kit. Instead, the components of the kit together are made available to the user, who can then use the components as needed to warm the ECG electrodes. For example, the adhesive may be used to bond the warmer 102 to the insert 120 and the ECG electrodes 104 may be removably connected to the insert 120, thus allowing the warmer 102 to warm the ECG electrodes 104.

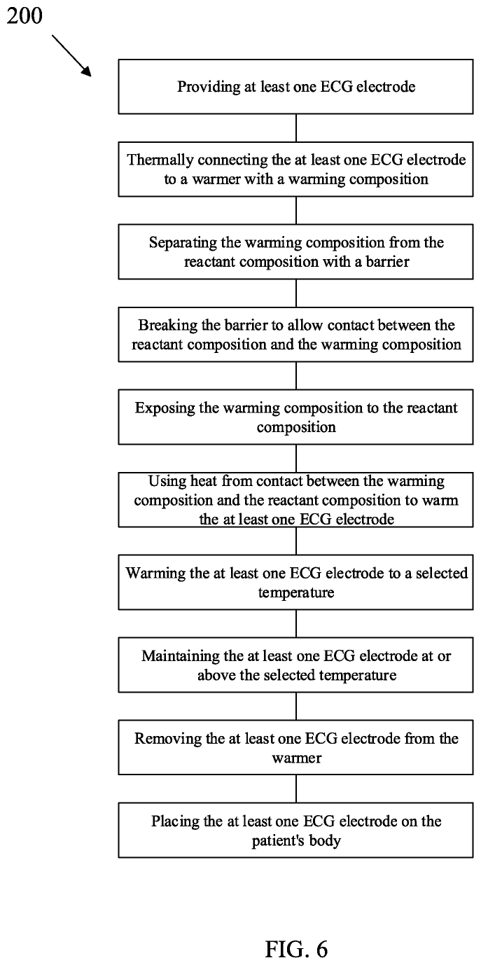

The present disclosure is also related to a method 200 of warming an electrocardiogram (ECG) electrode 104, illustrated in FIG. 6. Method 200 comprises providing ECG electrodes 104 as described above and thermally connecting the ECG electrodes 104 to a warmer 102 as described above. The method 200 may further comprise warming the provided ECG electrodes 104. The method 200 may be used to warm any number of ECG electrodes, including at least one ECG electrode 104, at least two ECG electrodes 104, a plurality of ECG electrodes 104, or six ECG electrodes 104, depending on the number of ECG electrodes 104 provided. Method 200 may further comprise removing the ECG electrodes 104 from the warmer 102 and/or placing the ECG electrodes 104 on the patient's body. Thus, method 200 may encompass providing the ECG electrode 104 and warmer 102, warming up the ECG electrode 104 with the warmer 102, removing the ECG electrode 104 from the warmer 102, and/or placing the ECG electrode 104 on the patient.

As mentioned above, the warmer 102 comprises an exothermic warming composition 114. Method 200 may further comprise exposing the warming composition 114 to the reactant composition 116, as disclosed above, to release heat, and may also comprise using the heat from contact between the warming composition 114 and the reactant composition 116 to warm the ECG electrodes 104. Method 200 may also comprise separating the warming composition 114 from the reactant composition 116 with the barrier 118, as discussed in more detail above. Additionally, method 200 may comprise breaking the barrier 118 to allow contact between the warming composition 114 and the reactant composition 116. As discussed above, in some embodiments, the warming composition 114 is air-activated and the reactant composition 116 is ambient air. In such an embodiment, the method 200 may also comprise oxidizing the warming composition 114.

The method 200 may comprise providing the warmer 102 encased within an envelope or providing both the warmer 102 and the ECG electrode 104 encased within the envelope. The ECG electrode 104 may be physically connected to the warmer 102, either directly or through an insert 120 as described above. The method 200 may also comprise warming the ECG electrode 104 to the selected temperature as described in detail above. By warming the ECG electrodes 104 to a comfortable temperature, the ECG electrodes 104 are not cold or uncomfortable when applied to the patient's body. The method 200 may comprise warming the ECG electrodes 104 to the selected temperature within the first time period and may comprise maintaining the ECG electrodes 104 at or above the selected temperature for the second time period. As mentioned above, the first time period and the second time period provide flexibility to the user by allowing the user to prepare the ECG electrodes 104 either when they are needed, knowing that the electrodes 104 will warm up within a reasonable amount of time (the first time period), or before they are needed, knowing that the electrodes 104 will remain warm for a specific amount of time (the second time period).

For some embodiments, the first time period is five minutes, and the method 200 comprises warming the ECG electrodes 104 to the selected temperature within five minutes. For some embodiments, the first time period is ten minutes, and the method 200 comprises warming the ECG electrodes 104 to the selected temperature within ten minutes. Other time periods may also be selected for the first time period. For some embodiments, the second time period is at least 45 minutes, and the method 200 comprises maintaining the ECG electrodes 104 at or above the selected temperature for at least 45 minutes. For some embodiments, the second time period is at least 90 minutes, and the method 200 comprises maintaining the ECG electrodes 104 at or above the selected temperature for at least 90 minutes. For some embodiments, the second time period is at least 120 minutes, and the method 200 comprises maintaining the ECG electrodes 104 at or above the selected temperature for at least 120 minutes. Other time periods may also be selected for the second time period. The selected temperature may be 110 degrees Fahrenheit, 120 degrees Fahrenheit, 130 degrees Fahrenheit, 140 degrees Fahrenheit, or any other temperature. It is desirable for the selected temperature to be high enough that the ECG electrodes 104 are warm to the touch for the patient.

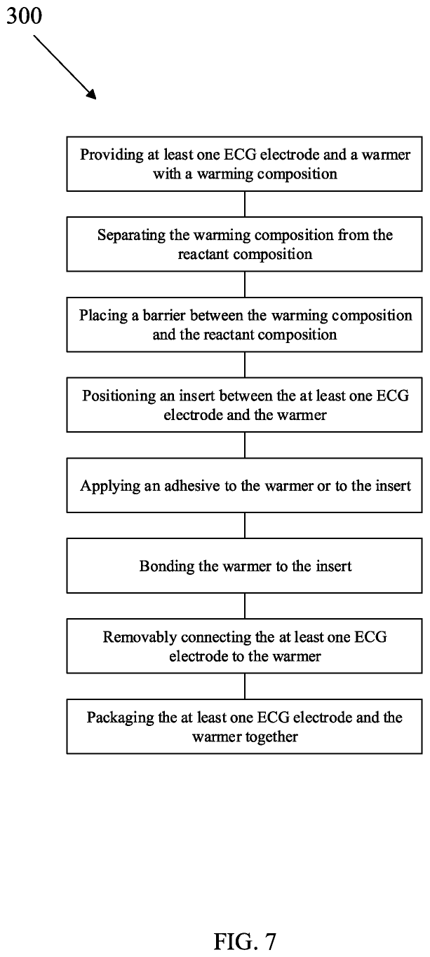

The present disclosure is also related to a method 300 of assembling the electrocardiogram (ECG) electrode warming system or kit 100, illustrated in FIG. 7. The method 300 comprises providing at least one ECG electrode 104 as disclosed above and a warmer 102 as disclosed above and packaging the at least one ECG electrode 104 and the warmer 102 together. Method 300 can involve any number of ECG electrodes, including at least one ECG electrode 104, at least two ECG electrodes 104, a plurality of ECG electrodes 104, or six ECG electrodes 104, depending on the number of ECG electrodes 104 provided. Method 300 may comprise additional components. For example, the method 300 may comprise removably connecting the ECG electrode 104 to the warmer 102, separating the warming composition 114 of the warmer 102 from the reactant composition 116, and/or placing a barrier 118 between the warming composition 114 and the reactant composition 116. As discussed above, the barrier 118 may be breakable by a user to allow the reactant composition 116 to contact the warming composition 114, and contact between the reactant composition 116 and the warming composition 114 may cause the warming composition 114 to release heat. The warming composition 114 may be air-activated and the reactant composition 116 may be ambient air. The method 300 may also comprise configuring the warmer 102 to warm the ECG electrode 104 when the barrier 118 is broken, encasing the warmer 102 in an envelope configured to separate the warmer 102 from ambient air, encasing the ECG electrode 104 in the envelope with the warmer 102, and/or preventing the warming composition 114 from oxidizing by separating the warming composition 114 from ambient air.

Method 300 may also comprise positioning the insert 120 between the ECG electrode 104 and the warmer 102 and/or removably connecting the ECG electrode 104 to the insert 120. Additionally, method 300 may comprise bonding the warmer 102 to the insert 120. Bonding the warmer 102 to the insert 120 may comprise applying an adhesive to the warmer 102 or to the insert 120, and applying an adhesive to the warmer 120 or to the insert 120 may comprise rolling the adhesive onto the warmer 102 or the insert 120 or spraying the adhesive onto the warmer 102 or the insert 120. As explained above, the adhesive may be a polyurethane glue.

The method 300 may also comprise packaging the warming composition 114 in a material that is permeable to the reactant composition 116 to facilitate contact between the warming composition 114 and the reactant composition 116 once the barrier 118 is removed or broken. The material used may also be selected to help prevent the warming composition 114 from passing through the material, while remaining permeable to the reactant composition 116. Because heavier fabrics may help in this regard, method 300 may comprise packaging the warming composition 114 in a fabric with a weight of at least 50 grams per square meter (GSM), at least 60 GSM, 70 GSM, 75 GSM or 80 GSM. The fabric may be a polypropylene fabric. Other materials and weights may also be implemented.

The method 300 may also comprise configuring the warmer 102 to warm the ECG electrode 104 to the selected temperature as described in detail above. By warming the ECG electrodes 104 to a comfortable temperature, the ECG electrodes 104 are not cold or uncomfortable when applied to the patient's body. The method 300 may comprise configuring the warmer 102 to warm the ECG electrodes 104 to the selected temperature within the first time period and may comprise configuring the warmer 102 to maintain the ECG electrodes 104 at or above the selected temperature for the second time period. As mentioned above, the first time period and the second time period provide flexibility to the user by allowing the user to prepare the ECG electrodes 104 either when they are needed, knowing that the electrodes 104 will warm up within a reasonable amount of time (the first time period), or before they are needed, knowing that the electrodes 104 will remain warm for a specific amount of time (the second time period). The selected temperature, the first time period, and the second time period are all dependent on the formulation of the warming composition 114 and/or the reactant composition 116. Thus, method 300 may also comprise selecting the ratios and amounts of the components of the warming composition 114 and/or the reactant composition 116.

For some embodiments, the first time period is five minutes, and the method 300 comprises configuring the warmer 102 to warm the ECG electrodes 104 to the selected temperature within five minutes. For some embodiments, the first time period is ten minutes, and the method 300 comprises configuring the warmer 102 to warm the ECG electrodes 104 to the selected temperature within ten minutes. Other time periods may also be selected for the first time period. For some embodiments, the second time period is at least 45 minutes, and the method 300 comprises configuring the warmer 102 to maintain the ECG electrodes 104 at or above the selected temperature for at least 45 minutes. For some embodiments, the second time period is at least 90 minutes, and the method 300 comprises configuring the warmer 102 to maintain the ECG electrodes 104 at or above the selected temperature for at least 90 minutes. For some embodiments, the second time period is at least 120 minutes, and the method 300 comprises configuring the warmer 102 to maintain the ECG electrodes 104 at or above the selected temperature for at least 120 minutes. Other time periods may also be selected for the second time period. The selected temperature may be 110 degrees Fahrenheit, 120 degrees Fahrenheit, 130 degrees Fahrenheit, 140 degrees Fahrenheit, or any other temperature. It is desirable for the selected temperature to be high enough that the ECG electrodes 104 are warm to the touch for the patient.

It will be understood that implementations of an ECG electrode warming package are not limited to the specific assemblies, devices and components disclosed in this document, as virtually any assemblies, devices and components consistent with the intended operation of an ECG electrode warming package may be used. Accordingly, for example, although particular ECG electrode warming packages, and other assemblies, devices and components are disclosed, such may include any shape, size, style, type, model, version, class, measurement, concentration, material, weight, quantity, and/or the like consistent with the intended operation of ECG electrode warming packages. Implementations are not limited to uses of any specific assemblies, devices and components; provided that the assemblies, devices and components selected are consistent with the intended operation of an ECG electrode warming package.

Accordingly, the components defining any ECG electrode warming package may be formed of any of many different types of materials or combinations thereof that can readily be formed into shaped objects provided that the materials selected are consistent with the intended operation of an ECG electrode warming package. For example, the components may be formed of: polymers such as thermoplastics (such as ABS, Fluoropolymers, Polyacetal, Polyamide; Polycarbonate, Polyethylene, Polysulfone, and/or the like), thermosets (such as Epoxy, Phenolic Resin, Polyimide, Polyurethane, Silicone, and/or the like), any combination thereof, and/or other like materials; glasses (such as quartz glass), carbon-fiber, aramid-fiber, any combination thereof, and/or other like materials; composites and/or other like materials; metals, such as zinc, magnesium, titanium, copper, lead, iron, steel, carbon steel, alloy steel, tool steel, stainless steel, brass, nickel, tin, antimony, pure aluminum, 1100 aluminum, aluminum alloy, any combination thereof, and/or other like materials; alloys, such as aluminum alloy, titanium alloy, magnesium alloy, copper alloy, any combination thereof, and/or other like materials; any other suitable material; and/or any combination of the foregoing thereof. In instances where a part, component, feature, or element is governed by a standard, rule, code, or other requirement, the part may be made in accordance with, and to comply under such standard, rule, code, or other requirement.

Various ECG electrode warming packages may be manufactured using conventional procedures as added to and improved upon through the procedures described here. Some components defining an ECG electrode warming package may be manufactured simultaneously and integrally joined with one another, while other components may be purchased pre-manufactured or manufactured separately and then assembled with the integral components. Various implementations may be manufactured using conventional procedures as added to and improved upon through the procedures described here.

Accordingly, manufacture of these components separately or simultaneously may involve extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled with one another in any manner, such as with adhesive, a weld, a fastener (e.g. a bolt, a nut, a screw, a nail, a rivet, a pin, and/or the like), wiring, any combination thereof, and/or the like for example, depending on, among other considerations, the particular material forming the components.

It will be understood that methods for manufacturing or assembling ECG electrode warming packages are not limited to the specific order of steps as disclosed in this document. Any steps or sequence of steps of the assembly of an ECG electrode warming package indicated herein are given as examples of possible steps or sequence of steps and not as limitations, since various assembly processes and sequences of steps may be used to assemble ECG electrode warming packages.

The implementations of an ECG electrode warming package described are by way of example or explanation and not by way of limitation. Rather, any description relating to the foregoing is for the exemplary purposes of this disclosure, and implementations may also be used with similar results for a variety of other applications employing an ECG electrode warming package.

What is claimed is:

1. An electrocardiogram (ECG) electrode warming system comprising:
a warmer having an exothermic warming composition;
at least one ECG electrode configured to attach to a patient's body to detect electric signals, wherein the at least one ECG electrode is thermally connected to the warmer;
an insert positioned between the at least one ECG electrode and the warmer, wherein the at least one ECG electrode is removably connected to the insert and the warmer is bonded to the insert with an adhesive; and
a barrier configured to separate the exothermic warming composition from a reactant composition, wherein the barrier is breakable by a user to allow the reactant composition to contact the exothermic warming composition and wherein contact between the reactant composition and the exothermic warming composition causes the exothermic warming composition to release heat.

2. The ECG electrode warming system of claim 1, wherein the warmer is configured to warm the at least one ECG electrode to at least a selected temperature within five minutes and maintain the at least one ECG electrode at or above the selected temperature for at least 45 minutes.

3. The ECG electrode warming system of claim 2, wherein the selected temperature is 120 degrees Fahrenheit.

4. The ECG electrode warming system of claim 1, wherein the exothermic warming composition is packaged within a fabric with a weight of at least 50 grams per square meter.

5. An electrocardiogram (ECG) electrode warming system comprising:
a warmer having an exothermic warming composition;
at least one ECG electrode configured to attach to a patient's body to detect electric signals, wherein the at least one ECG electrode is thermally connected to the warmer; and
a barrier configured to separate the exothermic warming composition from a reactant composition, wherein the barrier is breakable by a user to allow the reactant composition to contact the exothermic warming composition and wherein contact between the reactant composition and the exothermic warming composition causes the exothermic warming composition to release heat.

6. The ECG electrode warming system of claim 5, further comprising an insert positioned between the at least one ECG electrode and the warmer, wherein the at least one ECG electrode is removably connected to the insert.

7. The ECG electrode warming system of claim 5, wherein the warmer is bonded to the insert.

8. The ECG electrode warming system of claim 7, wherein the warmer is bonded to the insert with an adhesive.

9. The ECG electrode warming system of claim 5, wherein the barrier is an envelope configured to encase the warmer and the reactant composition is ambient air.

10. The ECG electrode warming system of claim 5, wherein the warmer is configured to warm the at least one ECG electrode to at least a selected temperature within five minutes and maintain the at least one ECG electrode at or above the selected temperature for at least 45 minutes.

11. The ECG electrode warming system of claim 10, wherein the selected temperature is 120 degrees Fahrenheit.

12. The ECG electrode warming system of claim 5, wherein the exothermic warming composition is packaged within a fabric with a weight of at least 50 grams per square meter.

13. An electrocardiogram (ECG) electrode warming system comprising:
a warmer having an exothermic warming composition; and
at least one ECG electrode configured to attach to a patient's body to detect electric signals, wherein the at least one ECG electrode is removably and thermally connected to the warmer.

14. The ECG electrode warming system of claim 13, wherein the exothermic warming composition is air-activated.

15. The ECG electrode warming system of claim 13, further comprising a barrier configured to separate the exothermic warming composition from a reactant composition, wherein the barrier is breakable by a user to allow the reactant composition to contact the exothermic warming composition.

16. The ECG electrode warming system of claim 15, wherein the barrier is an envelope configured to encase the warmer and the reactant composition is ambient air.

17. The ECG electrode warming system of claim 13, wherein contact between a reactant composition and the exothermic warming composition causes the exothermic warming composition to release heat.

18. The ECG electrode warming system of claim 13, further comprising an insert positioned between the at least one ECG electrode and the warmer, wherein the at least one ECG electrode is removably connected to the insert and the warmer is bonded to the insert.

19. The ECG electrode warming system of claim 13, wherein the warmer is configured to warm the at least one ECG electrode to at least a selected temperature within five minutes.

20. The ECG electrode warming system of claim 13, wherein the warmer is configured to maintain the at least one ECG electrode at or above a selected temperature for at least 45 minutes.
